(19) United States
(12) Patent Application Publication
(10) Pub. No.: US 2006/0041233 A1
(43) Pub. Date: Feb. 23, 2006

(54) Reclosable apparatus and method to secure an appliance to a person

(76) Inventor: Michael L. Bowen, Mansfield, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/196,230

(22) Filed: Aug. 3, 2005

Related U.S. Application Data
(60) Provisional application No. 60/602,403, filed on Aug. 18, 2004.

(51) Int. Cl. A61M 5/32

(52) U.S. Cl. 604/180

(57) ABSTRACT

Apparatus and method to secure an appliance to a person is provided. The apparatus may have a base that attaches to a selected portion of a person's skin or other desired surface. A tongue may extend from the base and wrap around the appliance. At least a portion of the tongue may be alternately engaged with and disengaged from the tongue to accommodate securing and releasing the associated apparatus.

### RELATED APPLICATION

This application claims the benefit of U.S. Provisional Application No. 60/602,403, filed Aug. 18, 2004, the contents of which are hereby incorporated by reference in their entirety.

### TECHNICAL FIELD

The present invention is related in general to apparatus and methods associated with securing appliances to a person and more specifically to an apparatus and method to removably secure appliances or devices to a person's skin or other surfaces.

### BACKGROUND OF THE INVENTION

Various medical devices, or portions thereof, often include generally tubular structures, such as catheters, intravenous tubing, and various types of electrical and fiber optic conductors. Many times there is a need to secure a device to or near a patient's body so that the device can appropriately function. For example, catheters are often used in medical procedures for transporting various types of fluids to and from the body of a patient. These devices generally include long, tubular conduits made from flexible materials and extend from the patient to a nearby, stationary receptacle or source of fluid. Therefore, it is often necessary to secure a catheter tube near an insertion point on a patient's body to ensure proper placement and functioning as well offering some degree of comfort and mobility to a patient.

Practitioners initially used strips of conventional, medical grade adhesive tape to secure medical devices directly to the patient's skin. This technique often proved unsuccessful. Tape tends to become loose each time an associated device is adjusted or temporarily removed. Also, frequently removing adhesive tape from a patient's skin may cause irritation and discomfort.

Various types of catheter or tubular holding devices have been developed to provide desired restraint and to overcome disadvantages associated with the use of strips of adhesive tape. For example, some conventional devices include a strap which encircles the limb of a patient. To adequately restrain an associated catheter or other tubular structure, the strap must be kept fairly tight, which may cause discomfort and restrict blood flow to the patient's limb. Since the strap may generally be applied only to a limb, practitioners are often limited as to the number and type of locations to which such catheter tube holders may be secured.

Another type of catheter or tubular holding device includes a segment of material having one side coated with adhesive which may be applied directly to the patient's skin. A central tab may extend from the segment of material with a fastener for securing a catheter or other tubular structure. Disadvantages associated with such devices include fasteners which function properly for only particular tube sizes. Hospitals may therefore be required to stock different sizes of securing devices in order to accommodate different tube diameters. In addition, these devices may suffer from an inability to adequately restrain an associated tube in its longitudinal direction.

Many catheter tube holders and other devices used to releasably secure appliances with a patient include hook and loop materials. Such materials are relatively expensive as compared with medical grade adhesive tape.

### SUMMARY OF THE INVENTION

In accordance with teachings of the present invention, an apparatus is provided for releasably securing an appliance on or adjacent a person. For some applications the apparatus may include a base and a flap or tongue attached to the base. The tongue may be wrapped around portions of an appliance and then attached to the base to releasably secure the appliance to the base. The base may be formed from a first layer of material. The tongue may be formed from a second layer of material disposed on the first layer. One or more of the layers may be formed from medical grade adhesive tape or any other type of generally flexible material compatible with placement on a person's skin. Some embodiments of the present invention may include multiple tongues to releasably secure one appliance or multiple appliances to a single base.

Technical benefits of the present invention include providing a reliable, relatively low cost device to releasably secure an appliance on a person or on a surface adjacent to the person. The device may be opened and closed (reclosable) multiple times. The present invention eliminates the use relatively high cost hook and loop materials associated with some conventional tube holders.

Further technical benefits of the present invention include providing simple, relatively low cost methods to manufacture a reclosable holding device from two or more layers of material such as medical grade adhesive tape. The present invention also includes relatively simple and easy to learn methods to attach an appliance to a person or on a surface adjacent to the person. The holding device may be opened and closed multiple times to adjust the position of an associated appliance relative to a person or to remove one appliance and attach another appliance. Holding devices and securing devices may be formed in accordance with teachings of the present invention to accommodate releasably securing appliances having a wide variety of sizes and shapes. The present invention is not limited to releasably securing tubular structures.

### BRIEF DESCRIPTION OF THE DRAWINGS

A more complete and thorough understanding of the present invention and advantages thereof may be acquired by referring to the following description taken in conjunction with the accompanying drawings, in which like reference numbers indicate like features, and wherein:

FIG. 1 is a schematic drawing showing a top or front plan view of one apparatus satisfactory for use in releasably securing an appliance in accordance with teachings of the present invention;

FIG. 2 is a schematic drawing showing a bottom or back plan view with portions broken away of the apparatus in FIG. 1;

FIG. 3 is a schematic drawing showing an isometric view of the apparatus of FIG. 1 prior to releasably attaching an appliance in accordance with teachings of the present invention;

FIG. 4 is a schematic drawing showing a top or front plan view of another apparatus satisfactory for use in releasably securing an appliance in accordance with teachings of the present invention;

FIG. 5 is a schematic drawing showing a bottom or back plan view with portions broken away of the apparatus in FIG. 4;

FIG. 6 is a schematic drawing showing an isometric view of the apparatus of FIG. 4 prior to releasably attaching an appliance in accordance with teachings of the present invention;

FIG. 7 is a schematic drawing showing an isometric view with portions broken away of a generally tubular appliance in the process of being releasably attached to a patient in accordance with teachings of the present invention;

FIG. 8 is a schematic drawing showing an isometric view with portions broken away of the generally tubular appliance in FIG. 7 releasably attached to the patient in accordance with teachings of the present invention;

FIG. 9 is a schematic drawing showing a top plan view of an apparatus having a pair of tongues which may be releasably engaged with a single appliance in accordance with teachings of the present invention; and

FIG. 10 is a schematic drawing showing a top plan view of an apparatus having a pair of tongues which may be releasably engaged with a respective first appliance and a respective second appliance in accordance with teachings of the present invention.

### DETAILED DESCRIPTION OF THE INVENTION

Various embodiments of the present invention and its advantages are best understood by reference to FIGS. 1-10 wherein like numbers refer to same and like parts.

Apparatus and methods incorporating teachings of the present invention may be satisfactorily used to releasably secure a wide variety of appliances to a person or surface adjacent to a person. Various features of the present invention may be described with respect to releasably securing medical appliances with a patient's skin or surfaces adjacent to a patient. However, apparatus and methods incorporating teachings of the present invention may also be satisfactorily used to releasably secure a wide variety of appliances to a person or surfaces adjacent to a person. Examples of such appliances include wires, fiber optic conductors or other conduits associated with communication equipment or monitor equipment. The present invention is not limited to medical appliances associated with treatment of patients. The present invention is not limited to attachment to a person's skin.

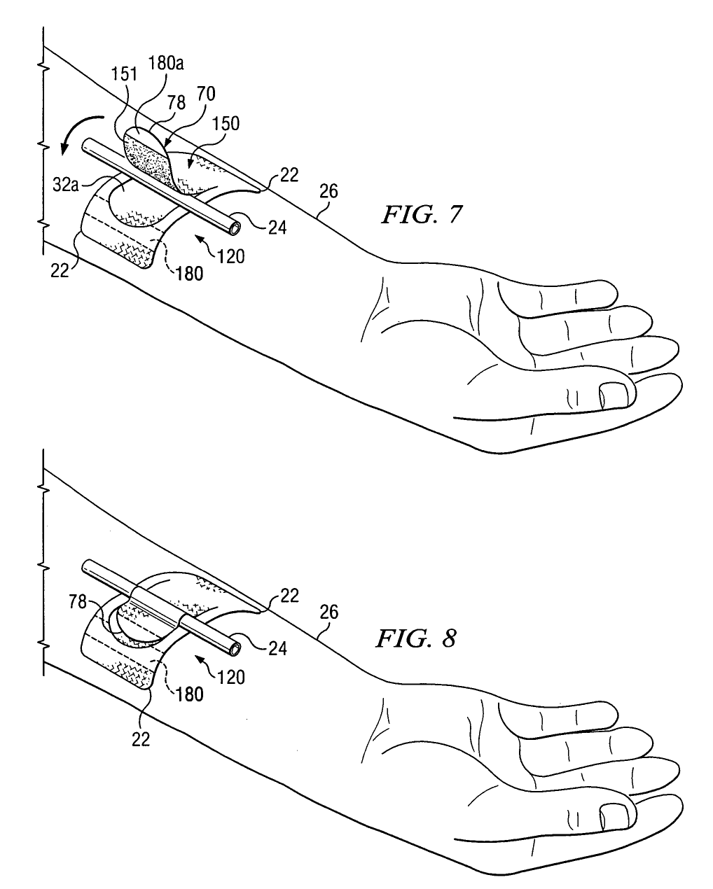

Various features of the present invention, will be described with respect to using apparatus such as reclosable holding devices 20, 120, 120a and 120b to releasably securing catheter tubes or other types of tubular members with a person's skin. See FIGS. 1, 4, 9 and 10. However, apparatus and methods incorporating teachings of the present invention may be satisfactorily used to releasably secure a wide variety of appliances including, but not limited to, lumens, tubes, wires, fiber optic conductors, communication equipment, monitoring equipment, feeding tubes, water tubes and oxygen tubes. Such appliances may be releasably secured to a person's skin (see FIGS. 7 and 8), clothing (not expressly shown) or a surface adjacent to a person (not expressly shown). Examples of such surfaces include, but are not limited to, hospital bed rail, desk, night stand, hospital gurney and surgery table.

FIGS. 1, 4, 9 and 10 show schematic top plan views of securing devices 20, 120, 120a and 120b which incorporate various teachings of the present invention. Securing devices 20, 120, 120a and 120b may also be described as general purpose releasable holding devices. For some specific applications securing devices 20, 120, 120a and 120b may be referred to as “reclosable tube holders” or “reclosable wire holders” as appropriate.

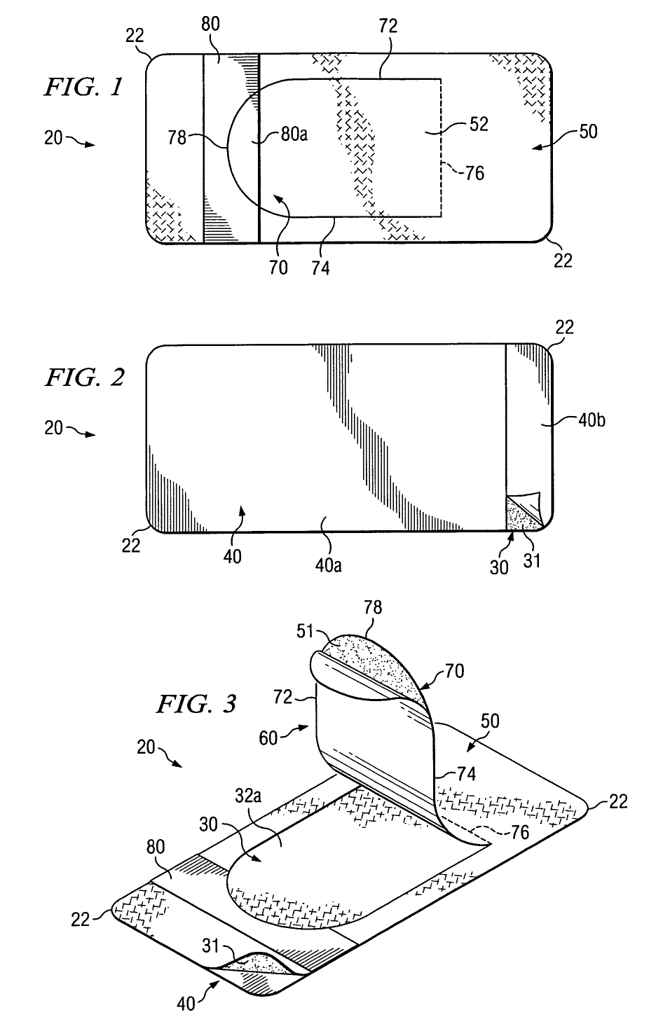

For embodiments such as shown in FIGS. 1, 2 and 3 securing device 20 may include two or more layers of material. For example, device 20 may include first layer or base 30 with first release liner 40 attached thereto. For some applications release liner 40 may be formed in two segments, 40a and 40b, to aid in removal of release liner 40 from base 30. See FIGS. 2 and 5. Securing device 20 may also include top layer 50 with second release liner 60 disposed therebetween. See FIG. 3. Release liners 40 and 60 may be formed from various types of material such as paper or plastic film. Release liners 40 and 60 may also be referred to as “removable liners”.

For embodiments such as shown in FIGS. 1, 2 and 3, securing device 20 may have a generally elongated rectangular configuration with generally rounded corners 22. Corners 22 cooperate with each other to reduce potential stress concentrations which might result in inadvertent peeling of base 30 when attached to a person or a surface adjacent to a person.

First layer or base 30 and second layer or top layer 50 may be formed from respective sheets or strips of medical grade adhesive tape. However, other materials may be satisfactorily used to form first layer 30 and second layer 50. Examples of such materials include but are not limited to medical grade foam, medical grade film and/or relatively thin, flexible sheets or strips of plastic.

Various types of medical grade adhesive tapes may be satisfactorily used to form first layer 30 and second layer 50. Examples of such adhesive tapes includes Durapore™ surgical tapes available from 3M Corporation. However, a wide variety of other medical grade adhesive tapes may be satisfactorily used. A wide variety of woven fabrics, non-woven fabrics, urethane foam and polyethylene films may also be satisfactorily used to form either first layer 30 and/or second layer 50. Other types of skin-friendly materials may also be satisfactorily used.

For many applications first layer 30 and second layer 50 may preferably be formed from breathable materials. For some applications material used to form second layer 50 may be stronger than material used to form first layer 30. Second layer 50 and particularly flap or tongue 70 may be formed from nonstretchable material. Material selections will depend upon intended use of the resulting holding device, desired length of time that the holding device will be in active use and cost associated with various materials.

Base 30 preferably includes first surface 31 and second surface 32. Top layer 50 may also include first surface 51 and second surface 52. First surface 31 of base 30 and first surface 51 of top layer 50 may be covered with various types of medical adhesive material. See FIGS. 2 and 3. First release liner 40 may be disposed on first surface 31 of base 30 to protect associated adhesive material prior to placement of base 30 on a patient's skin or a surface adjacent to a patient.

For some applications, a second release liner 60 may be disposed between portions of base 30 and top layer 50. See FIG. 3. Tongue or flap 70 may be formed from top layer 50 using various manufacturing techniques. Second release liner 60 may be used to protect base 30 during the formation of tongue or flap 70. For example, release liner 60 may be used when a rotary die kiss cutting process is used to form tongue 70 in second layer 50. Release liner 60 may prevent potential damage to adjacent portions of base 30 during cutting of tongue 70.

For some applications, tongue or flap 70 may have a generally elongated configuration defined in part by longitudinal edges 72 and 74 which extend generally parallel with each other from attached portion 76. Flap or tongue 70 may include a generally arcuate or curved end 78 disposed opposite from attached portion 76. Tongue 70 may be formed in top layer 50 by cutting edges 72, 74 and end 78. Attached portion 76 connects tongue 70 with top layer 50 and allows movement of tongue 70 relative to base 30. Attached portion 76 is indicated by dotted lines in FIGS. 1 and 3 for purposes of illustrating various features of the present invention. For some applications attached portion 76 may function similar to a hinge or flexible connection which allows rotation or lifting of tongue 70 relative to base 30 and placing tongue 70 over an appliance.

A flap or tongue may be formed in top layer 50 with a wide variety of configurations. The present invention is not limited to the configuration of flap 70. Also, flap 70 may be cut from a second layer of material and attached to base 30 (not expressly shown) without any other portions of the second layer of material being placed on or bonded with base 30. Various bonding techniques such as sonic welding or heat sealing may be used to secure portion 76 of flap 70 to second surface 32 of base 30.

For some applications, strip 80 may be placed on top layer 50 prior to forming tongue or flap 70. Strip 80 may be formed from paper or plastic film. During formation or cutting of tongue 70, segment 80a will be separated from strip 80. Segment or tab 80a disposed adjacent to end 78 provides a convenient location for grasping tongue 70. Segment or tab 80a may be particularly beneficial when trying to manipulate flap 70 while wearing gloves. Various types of advertising information and/or operating instructions may be printed on strip 80. Strip 80 and segment 80a may be used to identify end 78 of tongue 70 to assist in moving or lifting tongue 70 relative to base 30.

The portion of base 30 immediately adjacent to tongue 70 may be described as an attachment location for releasably securing an appliance with base 30. For the embodiments such as shown in FIGS. 3 and 6, the associated attachment location has been designated as 32a. For some applications tongue 70 and attachment location 32a may be approximately centered relative to second surface 32 of base 30.

The material used to form base 30 and particularly attachment location 32a along with adhesive material placed on first surface 51 of tongue 70 are preferably selected to allow multiple opening and closing of tongue 70 relative to base 30. Since attachment location 32a is generally protected from contact with a patient's skin and associated skin oils, tongue 70 may be engaged and disengaged multiple time with base 30. Adhesive material disposed on first surface 51 will generally prevent undesired movement of an attached appliance relative to base 30 and/or tongue 70. However, for some applications portions of the adhesive material on surface 51 may be removed (if desired) to prevent the adhesive material from contacting an associated appliance.

Various color combinations of materials may be used to form first layer 30 and second layer 50. Also, the adhesives placed on first layer 31 and first layer 51 may have different colors to indicate characteristics of the associated adhesive. For example, the adhesive used on first layer 31 may be compatible with attachment to a person's skin or may be compatible with attachment to clothing worn by a person. Each type of adhesive material may have a different color to indicate a respective type of use. Adhesive material placed on first surface 51 of tongue 70 may be selected for compatibility with attachment location 32a.

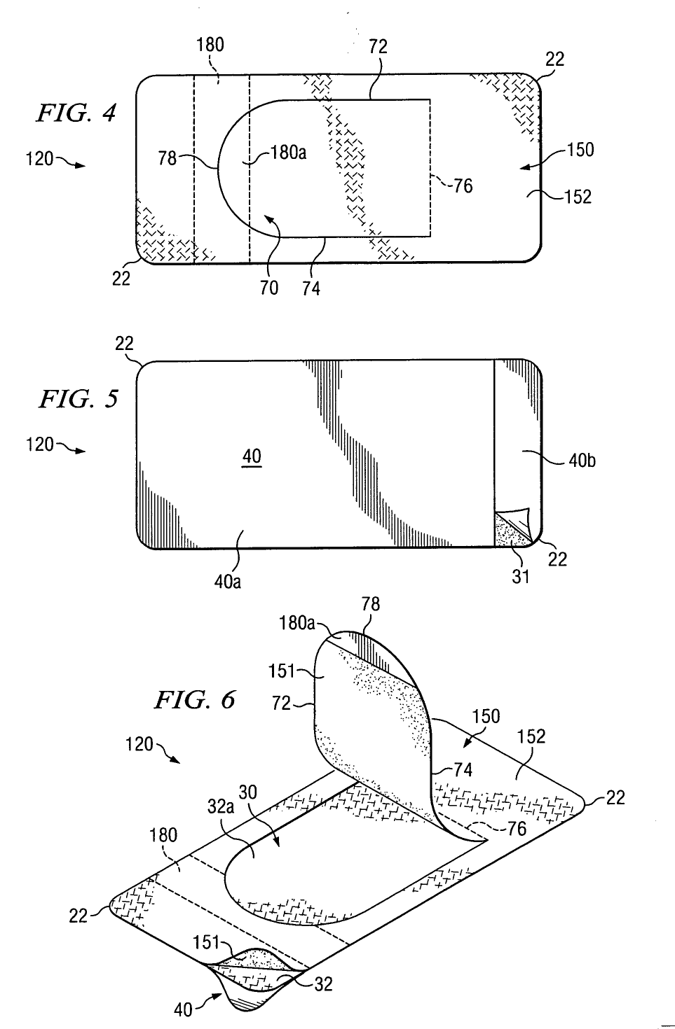

For embodiments such as shown in FIGS. 4, 5 and 6, securing device 120 may have a generally elongated, rectangular configuration with generally rounded corners 22. Securing device 120 may include some of the same features and benefits as previously described with respect to securing device 20. Securing device 120 may be formed with first layer or base layer 30 and release liner 40 as previously described with respect to device 20. However, second layer or top layer 150 of securing device 120 may be formed from material satisfactory for machining tongue 70 using kiss cutting techniques. Therefore, securing device 120 does not include second release liner 60 as previously described with respect to securing device 20.

Second layer or top layer 150 preferably includes first surface 151 and second surface 152. First surface 151 of top layer 150 may be covered with a wide variety of medical adhesive materials. For some applications the same adhesive material may be used on first surfaces 31 and 151. See FIG. 6.

Respective strips 180 of paper or plastic film may be disposed between top layer 150 and base 30 at approximately desired locations for end 78 of each tongue 70. Strip 180 may be formed from the same material as release liner 40 or any other suitable paper or plastic film. The materials used to form top layer 150 are preferably compatible with die rotary die kiss cutting cutting tongue 70 without damage to base 30. One example of such material includes 3M Durapore.

For some applications strip 180 may be disposed between top layer 150 and base 30 prior to forming tongue or flap 70. As a result during the formation or cutting of tongue 70, segment 180a of strip 180 will be separated from strip 180. Segment or tab 180a disposed adjacent to end 78 provides a convenient surface for grasping tongue 70. Segment or tab 180a may be particularly beneficial when trying to manipulate flap 70 while wearing gloves. Various types of advertising information and/or operating instructions may be printed on strip 180. Strip 180 and segment 180a may be used to identify end 78 of tongue 70 to assist in moving or lifting tongue 70 relative to base 30.

Materials used to form second layer or top layer 150 may be relatively translucent such that operating instructions and/or advertising material printed on strip 180 may be read by a person using associated securing device 120. As a result of forming tab or segment 180a, tongue 70 may be manipulated without engaging adhesive material disposed on other portions of first surface 151.

Securing devices 20 and 120 may be formed using various manufacturing techniques and procedures incorporating teachings of the present invention. One process associated with securing device 20 may include forming an elongated strip or blank with multiple layers of material (not expressly shown) defined in part by a first layer of release liner material, a second layer of material satisfactory for forming base 30, a third layer of release liner material and a fourth layer of material satisfactory for forming top layer 50. Respective strips 80 of paper or plastic film may be placed on the fourth layer of material (second surface 52 of layer 50) at locations corresponding approximately with desired locations for end 78 of each tongue 70.

One process associated with securing device 120 may include forming an elongated strip or blank with multiple layers of material (not expressly shown) defined in part by a first layer of release liner material, a second layer of material satisfactory for forming base 30, intermittent strips 180 disposed on the second layer of material (second surface 31 of base 30) and a third layer of material satisfactory for forming top layer 150. The first, second and third layers of material may extend generally longitudinally relative to each other. Strips 180 may be placed laterally between the second layer of material and the third layer of material at locations corresponding approximately with desired locations for end 78 of each tongue 70.

Preprinted instructions and/or advertising may be placed on one or more of the respective material rolls. The position of the instructions or advertising on each material layer may be selected to correspond with each securing device formed therefrom. Also, rolls of material with different colors may be used. For example, different colors may be used to indicate different types of uses for the resulting securing device. Each strip or blank of multiple layers of material may be formed by a wide variety of manufacturing techniques such as feeding material from respective rolls of release liner and medical grade adhesive tape in sequential order to form the desired strip.

Various types of cutting and/or stamping procedures may be used to form devices 20 and 120 from associated blanks. For example, rotary die kiss cutting techniques (such as kiss cutting) may be used to cut device 120 from the respective strip of multiple layers of material and to cut tongue 70 in top layer 150 without damage to base 30.

Adhesive material on first surface 51 and first surface 151 is preferably compatible with the material used to form second surface 32 of associated base 30. For some applications, portions of first surface 51 and first surface 151 may not be covered with adhesive material to avoid undesired contact between the adhesive material and the associated appliance when respective tongue 70 releasably secures the appliance to associated base 30.

Various methods and techniques incorporating teachings of the present invention may be used to releasably secure an appliance with a patient using securing devices 20 or 120. For example, release liner portions 40a and 40b may be removed from first surface 31 of base 30. Forming liner 40 with segments 40a and 40b facilitates wearing gloves while applying device 20 or 120 to a patient's skin. Base 30 may then be placed at a desired location on a patient's skin or on a surface adjacent to the patient.

For securing device 20, segment or tab 80a provides a convenient location to grasp and lift tongue 70 relative to base 30. Release liner 60 may then be removed from first surface 51. See FIG. 3. An appliance such as a catheter tube or an electrical wire may be placed on attachment location 32a and releasably secured thereto by engaging end 78 and adjacent portions of first surface 51 of tongue 70 with attachment location 32a.

For securing device 120, segment or tab 180a provides a convenient location to grasp or lift tongue 70 relative to base 30. As previously noted, securing device 120 does not include release liner 60. See FIG. 6. An appliance such as a catheter tube or an electrical wire may be placed on attachment location 32a and releasably secured thereto by engaging adjacent portions of first surface 51 of tongue 70 with attachment location 32a. Segment or tab 180a also provides a convenient location to grasp or lift tongue 70 to release an associated appliance. Segment or tab 180a facilitates multiple opening and closing of securing device 120.

FIGS. 7 and 8 are schematic drawings showing various steps associated with using securing device 120 to releasably engage tubular structure 24 at a desired location on arm 26 of a patient. For some applications tubular structure 24 may be a catheter. After release liner 40 has been removed from base 30, first surface 31 of securing device 120 may be applied at the desired location on arm 26. Segment or tab 180a may be lifted from top layer or second layer 150 to lift tongue 70 and expose attachment location 32a. A portion of tubular structure 24 may be placed on attachment location 32a. See FIG. 7. When tubular structure 24 is in the desired location, tab 180a may be used to releasably engage portions of first surface 151 of flap 150 with tubular structure 24 and attachment location 32a.

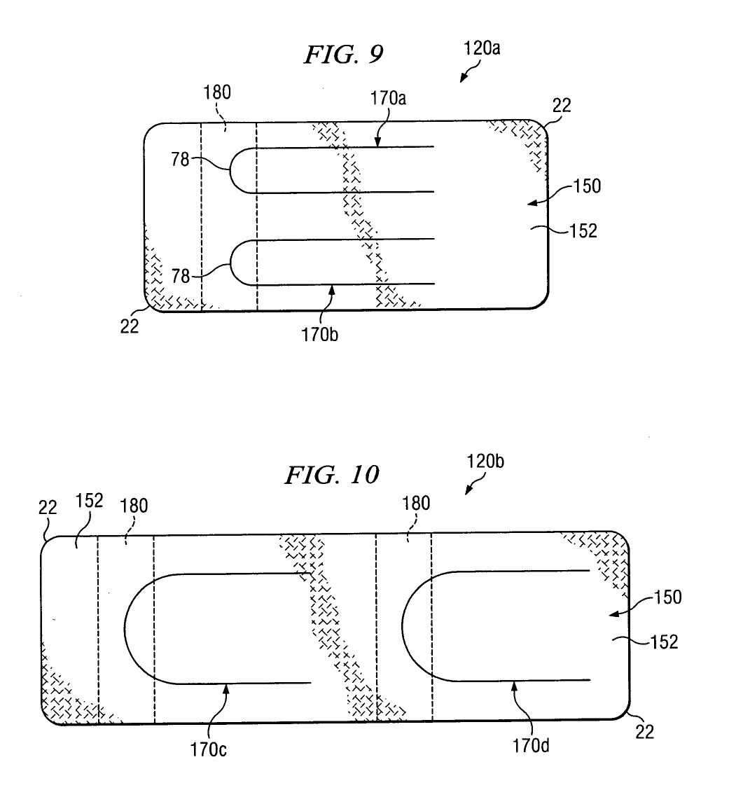

FIGS. 9 and 10 show alternative embodiments of the present invention. For the embodiment shown in FIG. 9, securing device 120a includes a pair of flaps or tongues 170a and 170b formed in second surface or top layer 150. Flaps 170a and 170b may be disposed generally parallel with each other. Flaps 170a and 170b may be used to releasably secure a single appliance with respective attachment locations 32a (not expressly shown).

For the embodiment shown in FIG. 10, securing device 120b includes tongues 170c and 170d formed in second layer or top layer 150. Tongues 170c and 170d may be disposed generally in series and aligned with each other along the length of top layer 150. Securing device 120b allows attaching multiple appliances such as two respective tubular structures 24 beneath respective tongues 170c and 170d (not expressly shown).

Although the present invention and its advantages have been described in detail, it should be understood that various changes, substitutions and alternations can be made herein without departing from the spirit and scope of the invention as defined by the following claims.

What is claimed is:

1. A device for releasably securing an appliance to a person comprising:
a first layer of material forming a base defined in part by a first surface and a second surface;
a tongue having a first surface and a second surface;
the tongue rotatably attached adjacent to the second surface of the base;
adhesive material respectively disposed on the first surface of the base and the first surface of the tongue; and
the tongue operable to releasably secure the appliance to the base when the first surface of the tongue engages the second surface of the base.

2. The device of claim 1 further comprising:
a second layer of material disposed on the second surface of the base layer; and
the tongue formed from a portion of the second layer of material.

3. The device of claim 1, further comprising a release liner disposed on the first surface of the base to protect the associated adhesive material.

4. The device of claim 1, further comprising:
a first removable release liner disposed on the first surface of the base to protect the associated adhesive material;
and a removable second release liner disposed between the first layer of material and the second layer of material; and
at least a portion of the second release liner disposed on the first surface of the tongue to protect the associated adhesive material prior to releasably securing the appliance to the base.

5. The device of claim 1, wherein the appliance comprises a tubular structure.

6. The device of claim 1, wherein the appliance comprises a tubular structure selected from the group consisting of a catheter tube, a wire for conducting electrical signals and a fiber optic conduit.

7. The device of claim 1, further comprising the first layer of material formed from medical grade adhesive tape.

8. The device of claim 1, further comprising the first layer of material selected from the group consisting of woven fabric, non-woven fabric, polyethylene, film, tape and combinations thereof.

9. The device of claim 1, further comprising the tongue cut from the second layer of material.

10. The device of claim 11 further comprising the base having a generally elongated rectangular configuration.

11. The device of claim 1 further comprising a first tongue and a second tongue disposed generally parallel with each other on the base.

12. The device of claim 1 further comprising:
a first tongue and a second tongue disposed on the base; and
the first tongue and the second tongue offset from each other to accommodate the first tongue releasably securing a first appliance with the base and the second tongue releasably securing a second appliance with the base.

14. A device for securing a medical appliance to a patient comprising:
a first layer of material forming a base having a first surface and a second surface;
a second layer of material disposed on the second surface of the base;
the second layer of material having a first surface and a second surface;
a flap formed from a portion of the second layer of material;
the flap having a first surface and a second surface;
the flap rotatably attached to the second layer of material;
adhesive material respectively disposed on the first surface of the base and the first surface of the flap; and
the flap operable to releasably secure the medical appliance to the base when the first surface of the flap engages adjacent portions of the second surface of the base.

15. The device of claim 14 further comprising the first layer of material and the second layer of material formed from medical grade adhesive tape.

16. The device of claim 14 further comprising a first flap and a second flap formed in the second layer of material.

17. The device of claim 14 further comprising:
a first release liner disposed on the first surface of the base; and
a second release liner disposed on the first surface of the flap.

18. The device of claim 14 further comprising the flap operable to secure a medical appliance selected from the group consisting of a catheter tube, an oxygen tube, a fiber optic conduit, and wires for conducting electrical signals.

19. The device of claim 14 further comprising:
the base having a generally elongated rectangular configuration;
the flap having a generally elongated rectangular configuration with dimensions smaller than the base; and
one end of the flap having a generally curved edge.

20. A method of securing an appliance to a person comprising:
attaching a base to a selected portion of the person's skin;
lifting a tongue disposed on and attached to the base to expose an attachment location on the base;
placing at least one portion of the appliance on the attachment location;
placing the tongue over the at least one portion of the appliance; and
releasably engaging adhesive material disposed on one surface of the tongue to the attachment location to releasably secure the appliance to the base.

21. The method of claim 20, further comprising removing a release liner from the base prior to attaching the base to the person's skin.

22. The method of claim 20, comprising lifting the tongue by grasping a tab formed on one end of the tongue.

23. The method of claim 20, further comprising removing a release liner from one surface of the tongue after lifting the tongue.

24. A method of manufacturing a reclosable securing device comprising:
forming a base having a first surface with adhesive material disposed thereon and a second surface having an attachment location;
forming a top layer of material having a first surface with adhesive material disposed thereon and a second surface;
placing the top layer of material on the base with the first surface of the top layer disposed adjacent to the second surface of the base; and
forming a tongue in the top layer with the tongue disposed adjacent to the attachment location of the base.

25. The method of claim 24, further comprising cutting the tongue from the top layer.

26. The method of claim 24, further comprising forming the base and the top layer from material selected from the group consisting of woven fabric, non-woven fabric, polyethylene film, medical grade adhesive tape, medical grade foam and combinations thereof.

27. The method of claim 24, further comprising forming a tab on the tongue for use in moving the tongue relative to the attachment location on the base.
